(19) United States
(12) Patent Application Publication
(10) Pub. No.: US 2026/0027324 A1
(43) Pub. Date: Jan. 29, 2026

(54) CATHETER ASSEMBLY

(71) Applicant: Medtronic, Inc., Minneapolis, MN (US)

(72) Inventors: Kyle Francis Flanagan, Tuam (IE); Mark Desmond Reid, Galway (IE); Andrew Stephen English, Galway (IE)

(21) Appl. No.: 19/150,013

(22) PCT Filed: Jan. 16, 2024
(86) PCT No.: PCT/IB2024/050400

§ 371 Date: Jul. 22, 2025

Related U.S. Application Data
(60) Provisional application No. 63/481,833, filed on Jan. 27, 2023.

(51) Int. Cl. A61M 25/00

(52) U.S. Cl. CPC A61M 25/0014; A61M 2207/00

(57) ABSTRACT

In examples described herein, a catheter includes an insert configured to mechanically connect a member (e.g., a hub) to an elongated body (e.g., a catheter shaft). In some examples, the insert includes an insert proximal portion and an insert distal portion, and the member defines a member distal lumen and a member proximal cavity. The insert distal portion is configured to be inserted into the elongated body and the member distal lumen is configured to receive a portion of the elongated body. The member proximal cavity is configured to receive the insert proximal portion such that the insert proximal face and the member proximal face are aligned. In some examples, when the insert proximal portion is positioned in the member proximal cavity, the insert proximal portion is configured to anchor the insert within the member proximal cavity to limit proximal and distal movement of the insert relative to the member.

This application claims the benefit of U.S. Provisional Patent Application Ser. No. 63/481,833, filed Jan. 27, 2023, the entire content of which is incorporated herein by reference.

### TECHNICAL FIELD

This disclosure relates to medical devices including catheters.

### BACKGROUND

A medical catheter has been proposed for use with various medical procedures. For example, in some cases, a medical catheter may be used to access and treat defects in blood vessels, such as, but not limited to, lesions or occlusions in blood vessels. In some examples, hubs are used to connect catheters and other medical devices to peripheral equipment. Hubs may allow a clinician or other user to transmit force to the catheter by manipulating the hub, and allow for delivery of supplementary devices or fluids (e.g., contrast agent, a therapeutic agent, or saline) through the hub and into a catheter lumen.

### SUMMARY

In general, the disclosure describes devices, systems, and techniques related to medical catheters including members (e.g., hubs) connected to catheter shafts (also referred to herein as catheter bodies) using friction and interlocking structures. The disclosure also describes methods of assembling a catheter assembly including a shaft and a member using friction and interlocking designs.

In examples described herein, a catheter includes an insert configured to mechanically connect a member (e.g., a hub) to an elongated body (e.g., a catheter shaft). The insert is configured to connect the member to the elongated body using friction and interlocking structures, which may provide for a relatively simple assembly process and eliminate the need for other methods of joining a hub to a catheter shaft. For example, the member and the elongated body can be connected via the insert and without adhesives or injection molding assembly methods, which may reduce the variability, mess, cost, and complications associated with such methods. Additionally, using friction and interlocking designs may help reduce leaks associated with other assembly methods and may facilitate automated assembly processes.

In some examples, the member defines a member distal lumen, a member proximal cavity, and a member proximal face and the insert comprises an insert proximal portion and an insert distal portion. The insert proximal portion defines an insert proximal face. The insert distal portion is configured to be inserted into an elongated body, and the member proximal cavity is configured to receive the insert proximal portion such that the insert proximal face and the member proximal face are aligned. When the insert proximal portion is positioned in the member proximal cavity (e.g., when the member proximal cavity receives the insert proximal portion), the insert proximal portion is configured to anchor the insert within the member proximal cavity to limit proximal and distal movement of the insert relative to the member. In some examples, the insert and the member are configured to compress the elongated body between the insert and the member, thus creating a friction fit to secure the elongated body to the member. The insert and the member may include one or more other features to limit rotation or axial movement of the insert or the member relative to each other.

Friction and interlocking assembly methods may provide visual and tactile feedback during assembly. For example, the insert proximal face and a member proximal face being aligned upon assembly may provide an indication during assembly that the components have been assembled correctly. This alignment may provide a visual or tactile indication that the insert is properly positioned within the member, which may reduce incorrectly assembled parts as well as time spent during the assembly process ensuring correct assembly and special skills required to assemble the catheter.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating a cross-sectional view of an example catheter that includes a member mechanically connected to an elongated body with an insert.

FIG. 2A is a conceptual diagram illustrating a side view of an example insert used for mechanically connecting a member to an elongated body.

FIG. 2B is a conceptual diagram illustrating an end view of the example insert shown in FIG. 2A.

FIG. 2C is a conceptual diagram illustrating a cross-sectional side view of an example member, which is configured to be mechanically connected to an elongated body with an insert.

FIG. 3A is a conceptual diagram illustrating a side view of an example insert configured to mechanically connect a member to an elongated body.

FIG. 3B is a conceptual diagram illustrating a cross-sectional side view of an example member configured to be mechanically connected to an elongated body with an insert.

FIG. 4 is a conceptual diagram illustrating a cross-sectional side view of another example member configured to be mechanically connected to an elongated body with an insert.

FIG. 5 is a conceptual diagram illustrating a cross-sectional side view of another an example member configured to be mechanically connected to an elongated body with an insert.

FIG. 6A is a conceptual diagram illustrating a side view of another example insert configured to mechanically connect a member to an elongated body

FIG. 6B is a conceptual diagram illustrating an end view of the example insert shown in FIG. 6A.

FIG. 6C is a conceptual diagram illustrating a cross-sectional side view of an example member configured to be mechanically connected to an elongated body with the insert of FIG. 6A.

FIG. 7A is a conceptual diagram illustrating a side view of another example insert configured to mechanically connect a member to an elongated body.

FIG. 7B is a conceptual diagram illustrating an end view of the example insert shown in FIG. 7A.

FIG. 7C is a conceptual diagram illustrating a cross-sectional side view of an example member configured to be mechanically connected to an elongated body with the insert of FIG. 7A.

FIG. 8A is a conceptual diagram illustrating a side view of an example insert including a threaded portion.

FIG. 8B is a conceptual diagram illustrating a cross-sectional side view of an example member including a threaded portion, which may be mechanically connected to an elongated body with the insert of FIG. 8A.

FIG. 9 is a flow diagram illustrating an example technique of assembling a catheter with a proximal member and insert according to one or more examples of this disclosure.

Like reference characters denote like elements throughout the description and figures.

### DETAILED DESCRIPTION

In examples described herein, a catheter includes an elongated body (e.g., a catheter shaft, which can also be referred to as a catheter body) and a member (e.g., a catheter hub) mechanically connected to the elongated body via an insert configured to create a friction or interference fit with the member either directly or indirectly via the elongated body, which can be positioned radially between the insert and the member. In some examples, a method of assembling the catheter includes inserting an insert into an elongated body and then retracting a member in a proximal direction over a portion of the elongated body where the insert has been inserted into elongated body.

The inserts described herein are configured to mechanically connect a member to an elongated body using friction and interlocking structures. The member and the insert described herein may be configured in a variety of different ways to secure the member to the elongated body. In some examples, the insert or the member includes structural features (such as protrusions) configured to compress the elongated body between the insert and the member. In this way, the insert is configured to secure the member to the elongated body with a friction fit and without the use of adhesives or other injection molding assembly steps.

The member and the insert described herein may be configured in a variety of different ways to enable an interlocking design of the member and insert. For example, the member may define a cavity (e.g., a lumen) configured to receive a portion of the insert. In some examples, the interlocking design may limit the member or insert from moving distally or proximally relative to each other once the components of the catheter have been assembled. Similarly, the member and the insert may include features configured to limit or prevent rotation of the insert or the member relative to each other. In some examples, no adhesive is used to connect the insert and the member, and no adhesive is used between the member and the elongated body or between the insert and the elongated body. Eliminating the use of adhesives or injection molding assembly steps may provide for a relatively simple assembly process and reduce the variability, mess, cost, and complications associated with such methods. Additionally, using friction and interlocking designs may help reduce leaks associated with other assembly methods and may facilitate automated assembly processes.

Although the systems and techniques of this disclosure are primarily described in the context of securing a hub to the proximal end of a catheter shaft, the techniques described herein may similarly allow attachment of other components to an elongated body. For example, a splitter, stopper, extension, or other component may be joined to an elongated body using the techniques of this disclosure. Other examples elongated bodies may include, but are not limited to, medical leads, medical tubes (e.g., cannula), syringes, sheaths (e.g., introducer sheath), and the like.

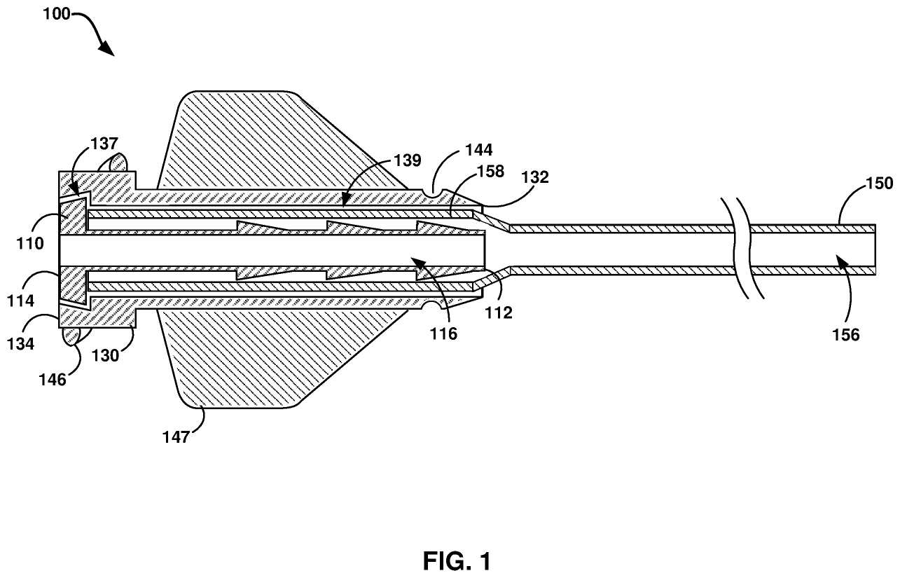

FIG. 1 is a conceptual diagram illustrating a cross-sectional view of an example catheter 100, the section view taken along a plane parallel to a longitudinal axis of catheter 100. Catheter 100 includes a member 130 mechanically connected to an elongated body 150 with an insert 110. Insert 110 is configured to secure member 130 to elongated body 150 via a friction or interference fit and without the use of adhesive. Member 130, which may be, for example, a catheter hub or another proximal catheter structure, defines a member distal end 132, a member proximal face 134, a member proximal cavity 137 and a member distal lumen 139. Member distal lumen 139 is configured to receive a portion (e.g., a proximal portion) of elongated body 150. Insert 110 defines an insert distal end 112, an insert proximal face 114, and an insert lumen 116 extending between insert distal end 112 and insert proximal face 114.

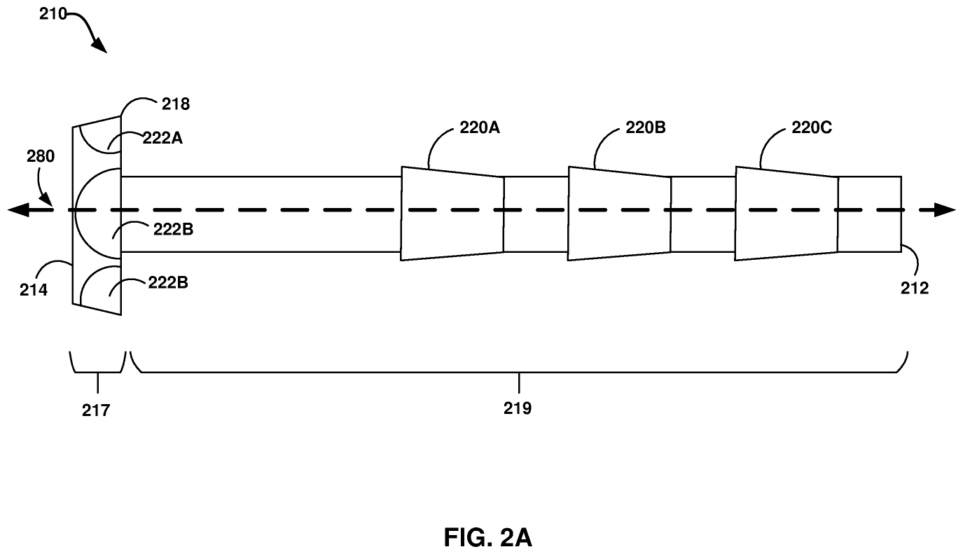

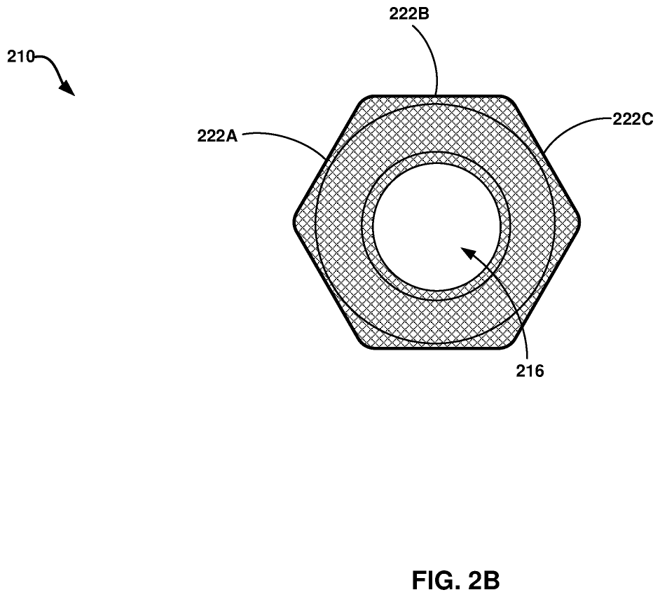

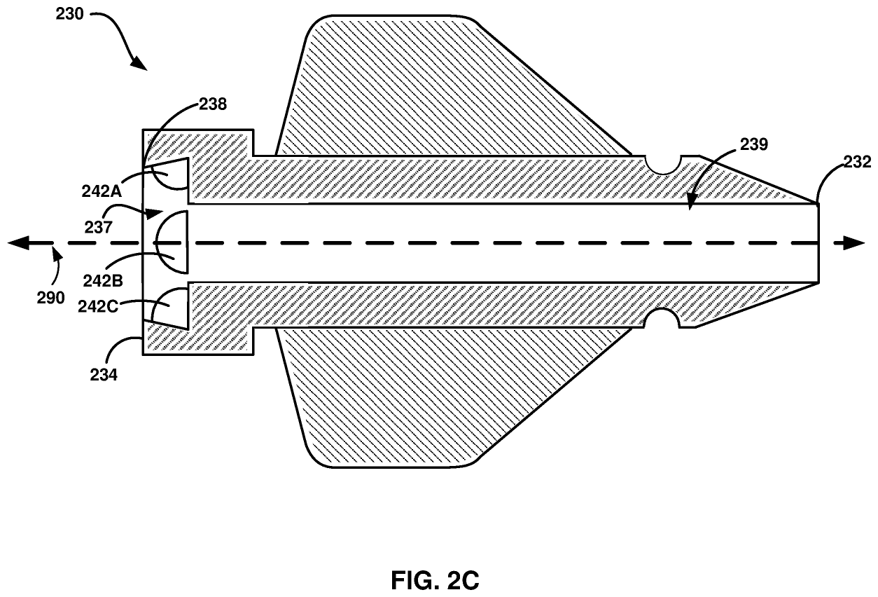

As shown in the example of FIG. 1, in some examples, when insert 110 is properly positioned (e.g., as designed and intended for use) within member 130, insert proximal face 114 and member proximal face 134 are aligned (e.g., flush or nearly flush to the extent permitted by manufacturing tolerances) at the proximal end of catheter 100. Insert 110 and member 130 are sized and shaped to facilitate this alignment between insert proximal face 114 and member proximal face 134 when an insert proximal portion 217 (FIG. 2A) of insert 110 is positioned within a member proximal cavity 237 (FIG. 2C) of member 130. The flush alignment of insert proximal face 114 and member proximal face 134 at the proximal end of catheter 100 may provide a visual or tactile indication that insert 110 is properly positioned within member 130 during assembly. The flush alignment of insert proximal face 114 and member proximal face 134 at the proximal end of catheter 100 may allow existing catheter bodies and hubs to be retrofitted to employ the features in this disclosure.

Elongated body 150 may be any suitable elongated body of catheter 100, such as, but not limited to a catheter shaft (also referred to as a catheter body in some examples). Elongated body 150 includes an elongated body wall 158 defining at least one lumen, such as an elongated body lumen 156 shown in FIG. 1. In the example of FIG. 1, elongated body wall 158 is compressed between insert 110 and member 130 when insert 110 is properly positioned within member 130. When insert 110 is properly positioned within member 130, movement of the insert 110, member 130, and elongated body 150 may be limited relative to each other. In this manner, member 130 may be secured to elongated body 150 via an interference or friction fit and without the use of adhesive, injection molding, or the like.

In some examples, when insert 110 is inserted into elongated body 150, insert lumen 116 is in fluid communication with elongated body lumen 156. Thus, a fluid injected into insert lumen 116 may be transferred through insert lumen 116 to elongated body lumen 156. In some examples, an inner diameter or inner dimension of elongated body 150 before insert 110 is inserted into elongated body 150 may the same or substantially similar to an inner diameter or inner dimension of insert 110 (or alternatively, the diameter or maximum dimension of insert lumen 116). In some examples, elongated body lumen 156 is configured to expand radially outwards when insert 110 is inserted into elongated body lumen 156 in order to accommodate insert 110.

Member 130 may include other various structural and/or functional features for use in medical procedures. For example, member 130 may include a luer thread 146 configured to facilitate connection of other devices to member 130. As another example, member 130 may include a plurality of grip elements 147, which may be in the shape of wings or flanges. Plurality of grip elements 147 may be configured to facilitate handling of catheter 100 via member 130. In addition, member may include a socket 144, which may be configured to facilitate attachment of a strain relief after mechanical connection of member 130 to the elongated body 150. Any of the aforementioned features of member 130 can be used alone or in combination with each other.

FIGS. 2A-2C illustrate various features of the example catheter components shown in FIG. 1, and may refer to the components of catheter 100 as shown in FIG. 1. Features described in relation to components shown in any of FIGS. 2A-2C may refer to features shown in other figures of FIGS. 2A-2C. FIG. 2A is a conceptual diagram illustrating a side view of an example insert 210 configured to mechanically connect a member 230 to an elongated body, such as elongated body 150. Insert 210 and member 230 are examples of insert 110 and member 130 of FIG. 1.

FIG. 2B is a conceptual diagram illustrating a proximal end view of insert 210 and illustrates one or more features of insert 210 configured to limit rotation of insert 210 relative to member 230 when insert 210 is properly positioned within in member 230 (e.g., as illustrated in FIG. 1, where insert proximal portion 217 is inserted within a member proximal cavity 237 and an insert proximal face 214 and a member proximal face 234 are aligned). Insert 210 defines an insert lumen 216.

FIG. 2C is a conceptual diagram illustrating a cross-sectional view of member 230, the cross-section being taken along a plane parallel to a member central longitudinal axis 290 through the center of member 230. Member 230, which may be, for example, a catheter hub, defines a member distal end 232, a member proximal face 234, a member distal lumen 239, and a member proximal cavity 237. Member distal lumen 239 is configured to receive a portion of elongated body 150.

In the example of FIG. 2A, insert 210 includes an insert proximal portion 217 and an insert distal portion 219. In some examples, insert proximal portion 217 generally defines a conical frustrum shape. In some examples, insert proximal portion 217 tapers in a proximal direction, e.g., between a larger distal diameter (or other maximum distal cross-sectional dimension in the case of a proximal portion 217 having a non-circular cross-sectional shape) and a smaller proximal diameter (or other maximum proximal cross-sectional dimension in the case of a proximal portion 217 having a non-circular cross-sectional shape).

Insert distal portion 219 may generally define a tubular or cylindrical shape, though it need not be perfectly circular in cross-section in all examples. Insert distal portion 219 is configured to be inserted into elongated body 150. In some examples, insert distal portion 219 defines an outer diameter (e.g., where insert distal portion 219 is circular or nearly circular in cross-section) or other maximum outer dimension (e.g., where insert distal portion 219 is non-circular in cross-section) which is slightly larger than an inner diameter (e.g., where elongated body 150 is circular or nearly circular in cross-section) or maximum inner dimension (e.g., where elongated body 150 is non-circular in cross-section) of elongated body 150 to create a secure fit when insert distal portion 219 is inserted into elongated body 150.

Insert 210 defines an insert distal end 212 and an insert proximal face 214. In the example shown in FIG. 2A, insert 210 includes a plurality of insert protrusions 220 (shown individually as insert protrusion 220A, insert protrusion 220B, and insert protrusion 220C). Plurality of insert protrusions 220 define, for example, surface irregularities of insert 210 that extend radially away from insert central longitudinal axis 280 of insert 210. In some examples, at plurality of insert protrusions 220, insert 210 defines an outer diameter (or other maximum outer dimension that is slightly larger than an inner diameter or inner dimension of elongated body 150) to create a secure interference fit when insert distal portion 219 is inserted into elongated body lumen 156 of elongated body 150.

While “diameters” of insert 210, elongated body 150, and other structures described herein are primarily referred to herein, in other examples, the diameter may be another maximum cross-sectional dimension (e.g., the cross-section being taken in a direction orthogonal to a longitudinal axis of the respective structure) in the case of a particular structure having a non-circular cross-section.

Insert 210 and member 230 include structural features for limiting rotation of insert 210 relative to member 230 when insert 210 is properly positioned within in member 230 (e.g., as illustrated in FIG. 1, where insert proximal portion 217 is inserted within member proximal cavity 237 and insert proximal face 214 and member proximal face 234 are aligned). For example, when member proximal cavity 237 receives insert proximal portion 217 such that proximal portion 217 is positioned in member proximal cavity 237, the configuration (e.g., a size and/or shape) of member proximal cavity 237 and insert proximal portion 217 may be configured to limit rotation of insert 210 relative to member 230. For example, where a clinician or other user provides a rotational force or torque to member 230, rotation of member 230 relative insert 210 or elongated body 150 may be limited due to surfaces of insert 210 and member 230 that interact to limit or prevent further relative rotation. Therefore, providing a rotational force or torque to member 230 causes a corresponding torque to insert 210 and elongated body 150, which may aid in navigation of elongated body 150 through the body of a patient.

FIGS. 2A-2C illustrate mating or interlocking features of insert 210 and member 230 that help limit rotation of insert 210 and member 230 relative to each other. In particular, in the example shown in FIG. 2A, and outer perimeter of insert proximal portion 217 defines a first plurality of flat sides 222 (shown individually in FIGS. 2A and 2B as flat side 222A, flat side 222B, and flat side 222C). In some examples, an inner perimeter of member proximal cavity 237 defines second plurality of flat sides 242 (shown individually in FIG. 2C as flat side 242A, flat side 242B, and flat side 242C). First plurality of flat sides 222 and second plurality of flat sides 242 are configured to limit rotation of insert 210 relative to member 230. For example, first plurality of flat sides 222 and second plurality of flat sides 242 may engage or interface to limit rotation of insert 210 relative to member 230. The limited rotation of insert 210 may be rotation around an insert central longitudinal axis 280 extending between insert distal end 212 and insert proximal face 214. Similarly, first plurality of flat sides 222 and second plurality of flat sides 242 may engage or interface to limit rotation of member 230 relative to insert 210. The limited rotation of member 230 may be rotation around a member central longitudinal axis 290 extending between member distal end 232 and member proximal face 234.

In some examples, although rotation of insert 210 and member 230 relative to each other may be limited, insert 210 and member 230 may be able to rotate a small amount relative to member 230 and insert 210, respectively (e.g., less than 5 degrees, such as less than 1, 2, 3, 4, or 5 degrees).

Where insert 210 includes first plurality of flat sides 222 to limit rotation relative to member 230, first plurality of flat sides 222 may include any suitable number of flat sides. In the example of FIGS. 2A and 2B, first plurality of flat sides 222 includes six flat sides, but can include one, two, three, four, five, or more than six sides in other examples. In some examples, first plurality of flat sides 222 are arranged such that insert proximal portion 217 defines a hexagonal conical frustrum. In some examples, first plurality of flat sides 222 may extend around an entire outer perimeter (e.g., an outer circumference) of insert proximal portion 217. In other examples, first plurality of flat sides 222 may extend only partially around the outer perimeter of insert proximal portion 217.

In examples in which member 230 includes second plurality of flat sides 242 to limit rotation relative to insert 210, second plurality of flat sides 242 may include any suitable number of flat sides. Second plurality of flat sides 242 may include a number of sides corresponding to the first plurality of flat sides 222 of insert proximal portion 217. Second plurality of flat sides 242 may be arranged to interface with first plurality of flat sides 222 when insert 210 is properly inserted into member 230. As shown in the examples of FIGS. 2A-2C, second plurality of flat sides 242 includes six flat sides and first plurality of flat sides 222 includes six flat sides.

As shown in FIG. 2A, insert 210 includes features configured to compress a portion of elongated body 150 between insert 210 and member 230 to secure elongated body 150 to member 230 via a friction or interference fit. In some examples, insert 210 includes plurality of insert protrusions 220 (shown individually as insert protrusion 220A, insert protrusion 220B, and insert protrusion 220C) extending radially outward from insert distal portion 219. When insert distal portion 219 is inserted into elongated body 150 and when member distal lumen 239 receives elongated body 150, plurality of insert protrusions 220 compresses elongated body 150 between insert 210 and member 230. When plurality of insert protrusions 220 compresses elongated body 150 between insert 210 and member 230, plurality of insert protrusions 220 helps reduce relative proximal and distal movement between member 230 and elongated body 150.

In addition, when plurality of insert protrusions 220 compresses elongated body 150 between insert 210 and member 230, plurality of insert protrusions 220 creates a seal, which can be a fluid tight or nearly fluid tight seal (to the extent permitted by manufacturing tolerances) between insert 210 and member 230. In this manner, when plurality of insert protrusions 220 compresses elongated body 150 between insert 210 and member 230, plurality of insert protrusions 220 is configured to prevent a fluid from traveling through member 230 in a radial space between elongated body 150 and member 230. In some examples, one or more seals (e.g., O-ring, or other rubber seal), are placed between insert 210 and member 230 to further prevent a fluid from traveling in a radial space between elongated body 150 and member 230. A seal may be placed in member proximal cavity 237, or in member distal lumen 239. In some examples, a seal (e.g., O-ring, or other rubber seal) may be placed between insert 210 and member 230 distal of plurality of insert protrusions 220 or proximal of plurality of insert protrusions 220.

Although the example of FIG. 2A shows plurality of insert protrusions 220 as having three insert protrusions, plurality of insert protrusions 220 can include any number of insert protrusions. Each insert protrusion of the plurality of insert protrusions 220 may be a uniform shape, or each insert protrusion of the plurality of insert protrusions 220 may have a unique shape, or some insert protrusion of the plurality of insert protrusions 220 may be uniform while others have a unique shape.

Plurality of insert protrusions 220 may take various shapes and orientations around insert 210. For example, plurality of insert protrusions 220 may extend around the entire outer circumference of insert 210, or plurality of insert protrusions 220 may only extend partially around the circumference of insert 210. Plurality of insert protrusions 220 may take the form of a taper or wedge, and may taper between a smaller distal diameter or dimension and a larger proximal diameter or dimension. In some examples, plurality of insert protrusions 220 may be positioned proximate insert distal end 212. In some examples, plurality of insert protrusions 220 may be positioned proximate insert proximal face 214 or insert proximal portion 217. In some examples, plurality of insert protrusions 220 may be positioned proximate a middle point between insert distal end 212 and insert proximal face 214. Plurality of insert protrusions 220 may have uniform spacing between each insert protrusion, or may have non-uniform spacing between each insert protrusion.

Plurality of insert protrusions 220 may include a material the same as insert 210, or may include a different material as insert 210. In examples where plurality of insert protrusions 220 include a different material as insert 210, plurality of insert protrusions 220 may include a material that is more compliant than a material of insert 210.

In the example of FIG. 2C, member proximal cavity 237 is configured to receive insert proximal portion 217. When member proximal cavity 237 receives insert proximal portion 217 (e.g., when proximal portion 217 is positioned in member proximal cavity 237), insert proximal face 214 and member proximal face 234 are aligned and insert proximal portion 217 is configured to anchor insert 210 within member proximal cavity 237 to limit proximal and distal movement of insert 210 relative to the member 230.

In some examples, insert proximal portion 217 defines a non-return edge 218. Non-return edge 218 is configured to limit proximal or distal movement of insert 210 relative to member 230 when member proximal cavity 237 receives insert proximal portion 217 and insert proximal portion 217 is properly positioned within member proximal cavity 237 (e.g., as shown in FIG. 1, where insert proximal face 114 and member proximal face 134 are aligned). Non-return edge 218 may extend radially outward from insert central longitudinal axis 280. In some examples, insert proximal portion 217 tapers proximally (e.g., from a larger distal dimension to a small proximal dimension) to define non-return edge 218, which may be a distal-most edge of insert proximal portion 217. Non-return edge 218 may extend fully or partially around insert proximal portion 217. Non-return edge 218 may be located on insert proximal portion 217 at the largest diameter (or maximum cross-sectional dimension) of insert proximal portion 217.

In some examples, member proximal cavity 237 defines a deflecting edge 238. Deflecting edge 238 may extend radially inward toward member central longitudinal axis 290. In some examples, member proximal cavity 237 tapers proximally (e.g., from a larger distal dimension to a small proximal dimension) to define deflecting edge 238. Deflecting edge 238 may be positioned proximate member proximal face 234. Deflecting edge 238 may be located at a smallest diameter (or maximum cross-sectional dimension) of member proximal cavity 237. Deflecting edge 238 may extend fully or partially around member proximal cavity 237.

In some examples, deflecting edge 238 is configured such that when member proximal cavity 237 receives insert proximal portion 217, deflecting edge 238 deflects to enable insertion of insert 210 into member 230 across non-return edge 218 (e.g., a snap fit). However, once non-return edge 218 passes across deflecting edge 238, deflecting edge 238 may deflect back to help limit proximal and distal movement of member 230 relative to insert 210. In some examples, due at least in part to the interaction of deflecting edge 238 and non-return edge 218, when member 230 is retracted over insert 210 during assembly, member 230 may be difficult to separate from insert 210, and insert 210 may be difficult to separate from member 230 without adversely impacting the structural integrity of member 230 and/or insert 210.

In some examples, member 230 is a single, continuous piece (e.g., a unitary body construction). In other examples, member 230 has a multi-piece (e.g., two-piece) design. For example, member 230 may include a first member piece and a second member piece that are configured to be coupled together to form member 230. As an example, the first member piece and the second member piece may be longitudinal halves of member 230 that mirror each other. In some examples, the first member piece extends between member proximal face 234 and member distal end 232. In some examples, the second member piece extends between member proximal face 234 and member distal end 232. The first member piece and the second member piece may be configured to be coupled together via features configured to secure two bodies together (e.g., snap fit features, adhesive, laser welding, reflow, etc.). in addition, in some examples, the first and second member pieces are connected together via a hinge (e.g., a clam shell design). In the example of FIG. 2C, the section view of member 230 illustrates an example first member piece or second member piece, if member 230 includes a multi-piece construction.

Insert 210 is made of any suitable material or combination of materials, including, but not limited to, a polymer and/or a metal. In examples in which insert 210 is made metal, insert 210 may be formed from a machining process (e.g., lathe, computer numerical control (CNC) machining, or the like). In examples in which insert 210 is made of a polymer material, insert 210 may be formed from an injection molding, three-dimensional (3D) printing, or another suitable process. In examples in which insert 210 is made of a suitable polymer material, insert 210 may be formed of a single polymeric material, or may be formed of multiple polymeric materials. For example, where insert 210 is formed of a polymeric material, plurality of insert protrusions 220 may include a polymeric material different than the rest of insert 210.

Member 230 is made of any suitable material or combination of materials, including, but not limited to, a polymer and/or a metal. Thus, in some examples, member 230 may be formed from an injection molding, 3D printing, or other suitable process. In some examples, as described in relation to some examples below, member 230 may be formed in a two-stage process in which member 230 is formed from different polymeric materials. The different polymeric materials may have a different compliance.

In some examples, insert 210 and member 230 are sized or dimensioned depending on the materials used and desired interference fit (e.g., with an elongated body, such as elongated body 150). For example, an interference overlap (or gap) of an inner dimension of member 230 relative to elongated body 150 may depend on the relative size and compressibility of the materials of insert 210, member 230, and elongated body 150. As another example, an interference overlap (or gap) of an outer dimension of insert 210 relative to elongated body 150 may depend on the relative size and compressibility of the materials of insert 210, member 230, and elongated body 150.

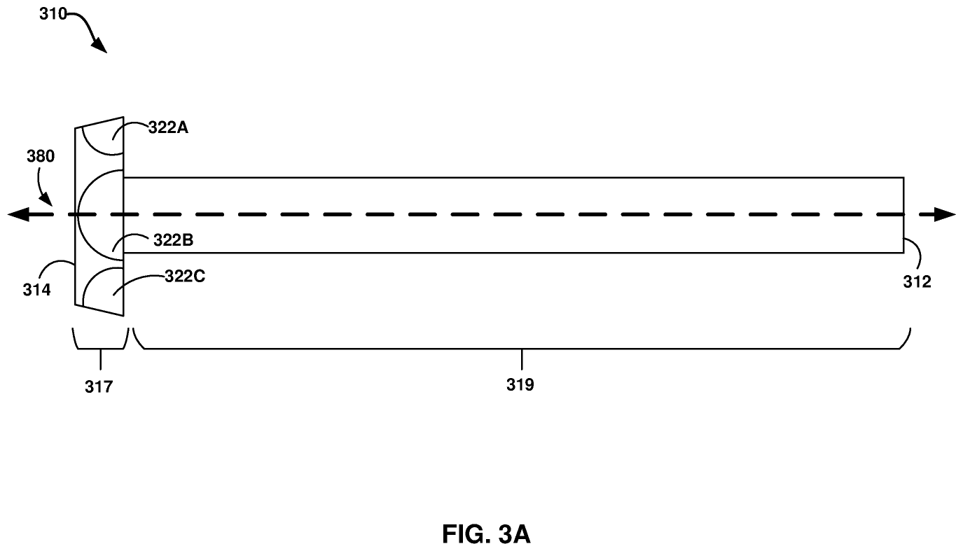

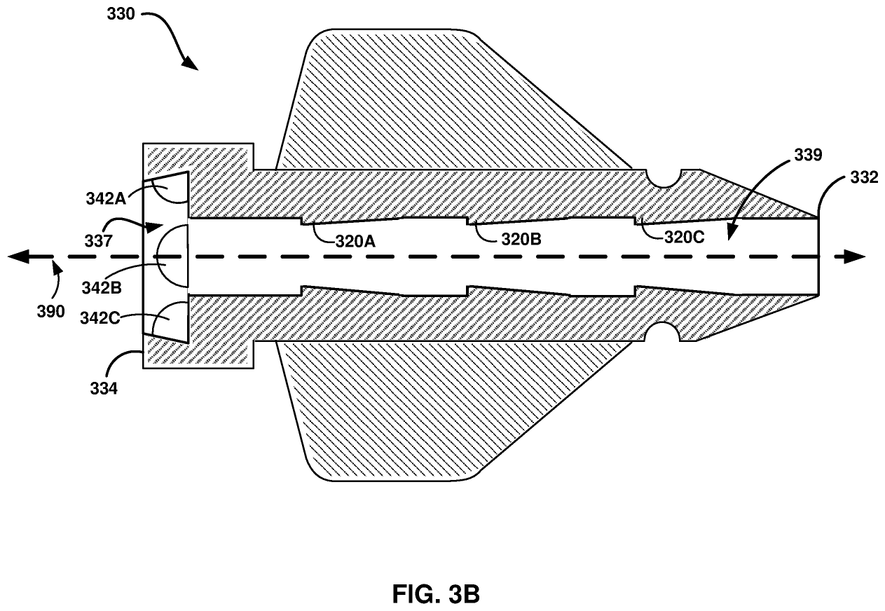

FIGS. 3A-3B shows other examples of catheter components and may refer to the components of catheter 100 as shown in FIG. 1. Features described in relation to components shown in any of FIGS. 3A-3C may refer to features shown in other figures of FIGS. 3A-3C. FIG. 3A is a conceptual diagram illustrating a side view of an example insert 310 configured to mechanically connect a member 330 to an elongated body, such as elongated body 150.

FIG. 3B is a conceptual diagram illustrating a cross-sectional view of member 330, the cross-section being taken along a plane parallel to a member central longitudinal axis 390 through the center of member 330. Member 330, which may be, for example, a catheter hub, defines a member distal end 332, a member proximal face 334, a member proximal cavity 337, and a member distal lumen 339. Member distal lumen 339 is configured to receive a portion of elongated body 150.

In the example of FIG. 3A, insert 310 defines an insert distal end 312 and an insert proximal face 314, and includes an insert proximal portion 317 and an insert distal portion 319. In some examples, insert proximal portion 317 generally defines a conical frustrum shape. For example, insert proximal portion 317 may taper in a proximal direction, e.g., between a larger distal diameter (or maximum cross-sectional dimension) and a smaller proximal diameter (or maximum cross-sectional dimension).

In some examples, insert distal portion 319 generally defines a tubular or cylindrical shape. Insert distal portion 319 is configured to be inserted into elongated body 150. In some examples, insert distal portion 319 defines an outer diameter that is slightly larger than an inner diameter of elongated body 150 to create a secure fit when insert distal portion 319 is inserted into elongated body 150. In other examples, insert proximal portion 317 and/or insert distal portion 319 can define other shapes.

FIGS. 3A-3B illustrate example mating or interlocking features of insert 310 and member 330 that help limit rotation of insert 310 and member 330 relative to each other in a similar way as illustrated and described in relation to FIGS. 2A-2C. In particular, in the example shown in FIG. 3A, an outer perimeter of insert proximal portion 317 defines a first plurality of flat sides 322 (shown individually in FIG. 3A as flat side 322A, flat side 322B, and flat side 322C). In some examples, an inner perimeter of member proximal cavity 337 defines a second plurality of flat sides 342 (shown individually in FIG. 3B as flat side 342A, flat side 342B, and flat side 342C) around member proximal cavity 337. Flat sides 322A, 322B, 322C of first plurality of flat sides 322 are configured to interact with corresponding flat sides 342A, 342B, 342C of second plurality of flat sides 342 to limit rotation of insert 310 relative to member 330. For example, first plurality of flat sides 322 and second plurality of flat sides 342 may interface to limit rotation of insert 310 relative to member 330. The limited rotation of insert 310 may be rotation around an insert central longitudinal axis 380 extending between insert distal end 312 and insert proximal face 314. Similarly, first plurality of flat sides 322 and second plurality of flat sides 342 may engage or interface to limit rotation of member 330 relative to insert 310. The limited rotation of member 330 may be rotation around a member central longitudinal axis 390 extending between member distal end 232 and member proximal face 234.

In some examples, although rotation of insert 310 and member 330 relative to each other may be limited, insert 310 and member 330 may be able to rotate a small amount relative to member 330 and insert 310, respectively (e.g., less than 5 degrees, such as less than 1, 2, 3, 4, or 5 degrees).

Where insert 310 includes first plurality of flat sides 322 to limit rotation relative to member 330, first plurality of flat sides 322 may include any number of flat sides. In the example of FIG. 3A, first plurality of flat sides 322 includes six flat sides, but can include one, two, three, or more sides in other examples. First plurality of flat sides 322 may be spaced such that insert proximal portion 317 defines a hexagonal conical frustrum. In some examples, first plurality of flat sides 322 may extend circumferentially around insert proximal portion 317. In some examples, first plurality of flat sides 322 may extend only partially around the circumference of insert proximal portion 317.

Where member 330 includes second plurality of flat sides 342 to limit rotation relative to insert 310, second plurality of flat sides 342 may include any suitable number of flat sides. Second plurality of flat sides 342 may include a number of sides corresponding to the first plurality of flat sides 322 of insert proximal portion 317. Second plurality of flat sides 342 may be arranged to interface with first plurality of flat sides 322 when insert 310 is properly inserted into member 330. As shown in the examples of FIGS. 3A-3B, second plurality of flat sides 342 includes six flat sides and first plurality of flat sides 322 includes six flat sides.

As shown in the example of FIG. 3B, member 330 includes structural features configured to compress a portion of elongated body 150 between insert 310 and member 330 to secure elongated body 150 to member 330 via a friction or interference fit and without requiring adhesive. In some examples, member 330 includes a plurality of member protrusions 320 (shown individually as member protrusion 320A, member protrusion 320B, and member protrusion 320C) extending radially inward into member distal lumen 339. Member protrusions 320 define surface irregularities such that the surface of member 330 defining member distal lumen 339 is not smooth.

When insert distal portion 319 is inserted into elongated body 150 and when member distal lumen 339 receives elongated body 150, plurality of member protrusions 320 is configured to compress elongated body 150 between insert 310 and member 330 to secure member 330 to elongated body 150 and limit or even prevent relative proximal and distal movement (along member central longitudinal axis 390) of elongated body 150 and member 330. When plurality of member protrusions 320 compresses elongated body 150 between insert 310 and member 330, plurality of member protrusions 320 creates a seal, which can be a fluid tight or nearly fluid tight seal (to the extent permitted by manufacturing tolerances), between insert 310 and member 330. In this manner, when plurality of member protrusions 320 compresses elongated body 150 between insert 310 and member 330, plurality of member protrusions 320 is configured to prevent a fluid from traveling through a radial space between member 330 and elongated body 150.

In some examples, one or more seals (e.g., O-ring, or other rubber seal), are placed between insert 310 and member 330 to further prevent a fluid from traveling through the radial space between member 330 and elongated body 150. A seal may be placed in member proximal cavity 337, or in member distal lumen 339. In some examples, a seal (e.g., O-ring, or other rubber seal) may be placed distal between insert 310 and member 330 distal of the plurality of member protrusions or proximal of plurality member protrusions.

Although the example of FIG. 3B shows plurality of member protrusions 320 as having three insert protrusions, plurality of member protrusions 320 can include any number of member protrusions. Each member protrusion of the plurality of member protrusions 320 may be a uniform shape, or each member protrusion of the plurality of member protrusions 320 may have a unique shape, or some member protrusion of the plurality of member protrusions 320 may be uniform while others have a unique shape.

Plurality of member protrusions 320 may take various shapes and orientations within member 330. For example, plurality of member protrusions 320 may extend around the entire inner circumference of member distal lumen 339, or plurality of member protrusions 320 may only extend partially around the inner circumference of member distal lumen 339. Plurality of member protrusions 320 may take the form of a taper or wedge, and may taper between a larger distal diameter or dimension and a smaller proximal diameter or dimension as shown in FIG. 3B. However, plurality of member protrusions 320 may also taper between a smaller distal diameter or dimension and a larger proximal diameter or dimension. In some examples, plurality of member protrusions 320 may be positioned proximate member distal end 332. In some examples, plurality of member protrusions 320 may be positioned proximate member proximal face 334 or member proximal cavity 337. In some examples, plurality of member protrusions 320 may be positioned proximate a middle point between member distal end 232 and member proximal face 334. Plurality of member protrusions 320 may have uniform spacing between each insert protrusion, or may have non-uniform spacing between each insert protrusion.

In the example shown in FIG. 3B, plurality of member protrusions 320 defines an inner diameter (e.g., where each insert protrusion of the plurality of member protrusions 320 is circular or nearly circular) or other maximum cross-sectional dimension (e.g., where each insert protrusion of the plurality of member protrusions 320 is non-circular) which is slightly smaller than an outer diameter or other maximum cross-sectional dimension of elongated body 150 (in some examples, as a portion of elongated is expanded by insert 310) to create a secure fit when member distal lumen 339 receives a portion of elongated body 150 and insert distal portion 319 has been inserted into elongated body 150.

In the example of FIG. 3B, member 330 and plurality of member protrusions 320 are formed of a single, continuous material. As such, member 330 may be formed from an injection molding, 3D printing, or other suitable process.

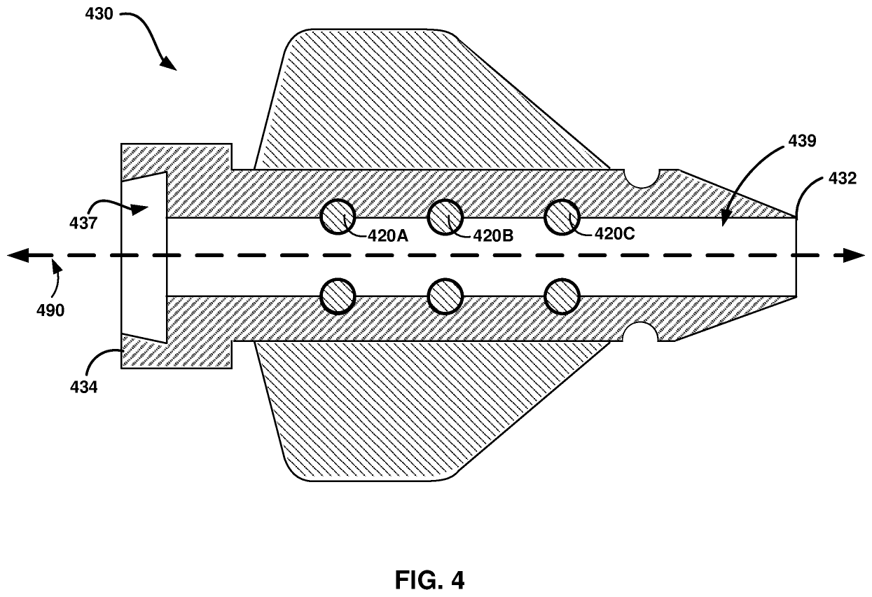

FIG. 4 is a conceptual diagram illustrating a cross-sectional view of a member 430, the cross-section being taken along a plane parallel to a member central longitudinal axis 490 through the center of member 430. Member 430, which may be, for example, a catheter hub, defines a member distal end 432, a member proximal face 434, a member proximal cavity 437, and a member distal lumen 439. Although not shown in the example of FIG. 4, member 430 may include features discussed in relation to member 130, member 230, and member 330 as discussed previously. In some examples, member 430 includes a plurality of member protrusions 420 (shown individually as member protrusion 420A, member protrusion 420B, and member protrusion 420C) extending radially inward into a member distal lumen 439. Plurality of member protrusions 420 may be formed and function similarly to the member protrusions described previously, except as noted herein.

Plurality of member protrusions 420 may be formed of a different, more complaint material as compared to a main body of member 430. For example, plurality of member protrusions 420 may include a rubberized polymer configured to compress. In some examples, member 430 and plurality of member protrusions 420 may be formed from a multi-step (e.g., two-step) injection molding process, where a first material is used to form plurality of member protrusions 420 and a second material is used to form the main body of member 430. In some examples, plurality of member protrusions 420 define a circular shape (e.g., such that an entire member protrusion of the plurality of member protrusions resembles an O-ring). In the example of FIG. 4, plurality of member protrusions 420 defines a circular or nearly circular cross section. Plurality of member protrusions 420 is configured to create a seal with an outer surface of elongated body 150, which can be a fluid tight or nearly fluid tight seal (to the extent permitted by manufacturing tolerances), when plurality of member protrusions 420 compresses elongated body 150 against an insert (e.g., insert 110, 210, 310). In this manner, plurality of member protrusions 420 configured to prevent a fluid from traveling through a radial space between elongated body 150 and member 430.

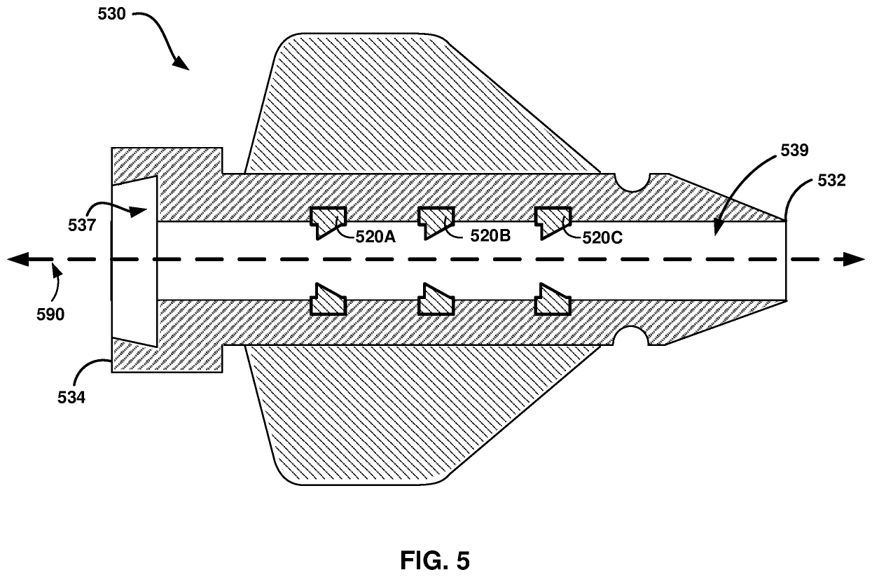

FIG. 5 is a conceptual diagram illustrating a cross-sectional view of a member 530, the cross-section being taken along a plane parallel to a member central longitudinal axis 590 through the center of member 530. Member 530, which may be, for example, a catheter hub, defines a member distal end 532, a member proximal face 534, a member proximal cavity 537, and a member distal lumen 539. Although not shown in the example of FIG. 5, member 530 may include features discussed in relation to member 130, member 230, member 330, and member 430 as discussed previously. In some examples, member 530 includes a plurality of member protrusions 520 (shown individually as member protrusion 520A, member protrusion 520B, and member protrusion 520C) extending radially inward into a member distal lumen 539. Plurality of member protrusions 520 may be formed and function similarly to the member protrusions described previously, except as noted herein.

Plurality of member protrusions 520 may be formed of a different, more complaint material as compared to a main body of member 530. For example, plurality of member protrusions 520 may include a rubberized polymer configured to compress. In some examples, member 530 and plurality of member protrusions 520 may be formed from a multi-step (e.g., two-step) injection molding process, where a first material is used to form plurality of member protrusions 520 and a second material is used to form the main body of member 530. In the example of FIG. 5, plurality of member protrusions 520 defines a cross section including a taper (e.g., wiper-like), in which plurality of member protrusions 520 taper from a larger distal internal diameter to a smaller proximal internal diameter.

Plurality of member protrusions 520 may be configured to deflect when a portion of elongated body 150 is inserted into member distal lumen 539. When plurality of member protrusions 520 deflects, plurality of member protrusions 520 may provide compression on elongated body 150 as plurality of member protrusions 520 remain deflected and apply a compressive force on elongated body 150. Because of the taper and direction of deflection of plurality of member protrusions 520, elongated body 150 may be limited from being removed from member 530 once elongated body 150 has been inserted into member 530 together with an insert (e.g., insert 110, 210, 310). Plurality of member protrusions 520 is configured to create a seal against an outer surface of elongated body 150 when plurality of member protrusions 520 compresses elongated body 150 against the insert (e.g., insert 110, 210, 310). The seal can be a fluid tight or nearly fluid tight seal (to the extent permitted by manufacturing tolerances). In this manner, plurality of member protrusions 520 are configured to prevent a fluid from traveling through a radial space between elongated body 150 and member 530.

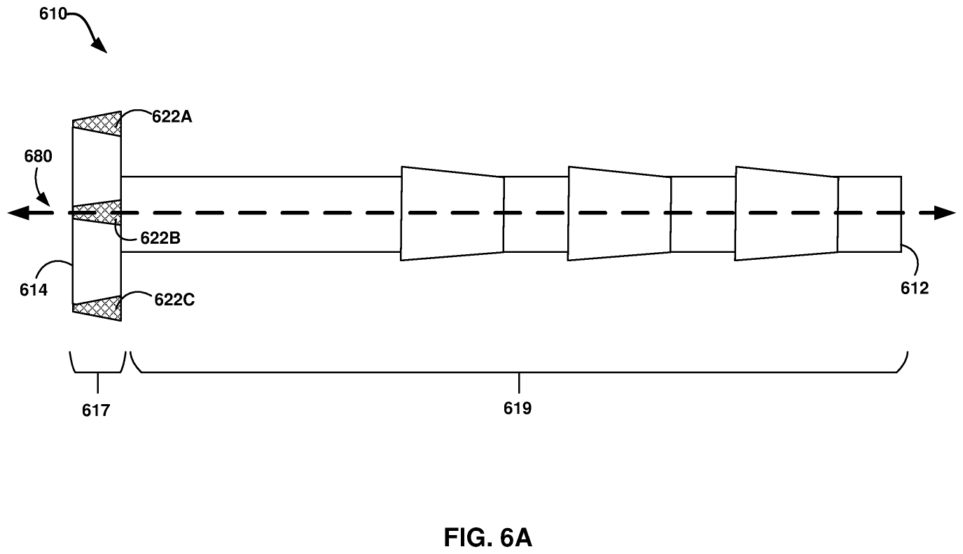

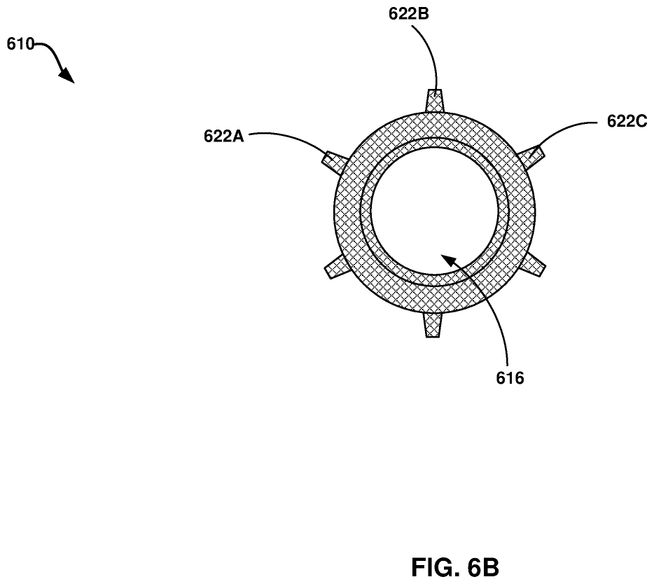

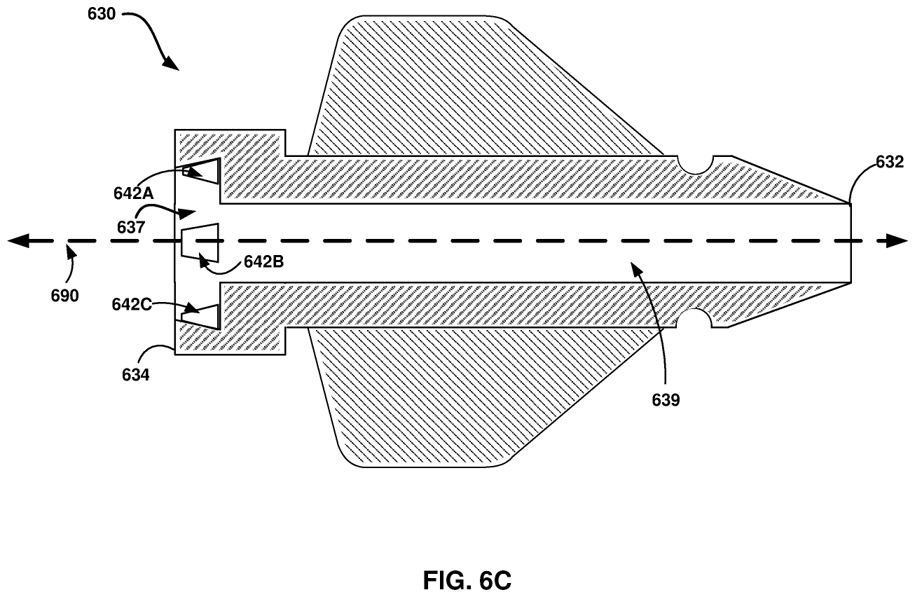

FIGS. 6A-6C illustrate various features of additional examples of the example catheter shown in FIG. 1, and may refer to the components of catheter 100 as shown in FIG. 1. Features described in relation to components shown in any of FIGS. 6A-6C may refer to features shown in other figures of FIGS. 6A-6C. FIG. 6A is a conceptual diagram illustrating a side view of an example insert 610 configured to mechanically connect a member 630 to an elongated body, such as elongated body 150. Insert 610 is another example of example insert 110 in FIG. 1, and insert 610 includes an insert proximal portion 617 and an insert distal portion 619.

FIG. 6B is a conceptual diagram illustrating a proximal end view of insert 610 and illustrates one or more features of insert 610 configured to limit movement of insert 610 relative to member 630 when insert 610 is properly positioned within in member 630 (e.g., as illustrated in FIG. 1, where insert proximal portion 617 is inserted within a member proximal cavity 637 and insert proximal face 614 and a member proximal face 634 are aligned). Insert 610 defines an insert lumen 616.

FIG. 6C is a conceptual diagram illustrating a cross-sectional view of a member 630, the cross-section being taken along a plane parallel to a member central longitudinal axis 690 through the center of member 630. Member 630, which may be, for example, a catheter hub, defines a member distal end 632, a member proximal face 634, a member distal lumen 639, and a member proximal cavity 637. Member distal lumen 639 is configured to receive a portion of elongated body 150. In the example of FIGS. 6A-6C, insert 610 and member 630 may be formed and function similarly to insert 210 and member 230 previously described in connection with FIG. 2, except as noted herein.

FIGS. 6A-6C illustrate another example of mating or interlocking features of insert 610 and member 630 to limit or even prevent rotation of insert 610 and member 630 relative to each other. In particular, in the example shown in FIG. 6A and FIG. 6B, insert proximal portion 617 of insert 610 includes a plurality of proximal portion protruding elements 622 (shown individually in FIGS. 6A and 6B as proximal portion protruding element 622A, proximal portion protruding element 622B, and proximal portion protruding element 622C) around the outer perimeter of insert proximal portion 617. In the example shown in FIG. 6C, member 630 defines a plurality of proximal cavity recesses 642 (shown individually in FIG. 6C as proximal cavity recess 642A, proximal cavity recess 642B, and proximal cavity recess 642C) around member proximal cavity 637. Plurality of proximal portion protruding elements 622 and plurality of proximal cavity recesses 642 are configured to mate together to limit or even prevent rotation of insert 210 relative to member 630 about an insert central longitudinal axis 680 and/or member central longitudinal axis 690 (which can be coaxial with insert central longitudinal axis 680 in some examples). That is, each proximal cavity recess 642A, 642B, 642C of plurality of proximal cavity recesses 642 is configured to receive a respective proximal portion protruding element 622A, 622B, 622C of plurality of proximal portion protruding elements 622 to rotationally fix insert 610 and member 630. Similarly, plurality of proximal cavity recesses 642 may be configured to receive plurality of proximal portion protruding elements 622 to limit rotation of member 630 relative to insert 610.

In some examples, although rotation of insert 610 and member 630 relative to each other may be limited, insert 610 and member 630 may be able to rotate a small amount relative to member 630 and insert 610, respectively (e.g., less than 5 degrees, such as less than 1, 2, 3, 4, or 5 degrees) due at least in part to a small tolerance between proximal cavity recesses 642 and proximal portion protruding elements 622.

Plurality of proximal portion protruding elements 622 may include any suitable number of proximal portion protruding elements. In the example of FIGS. 6A and 6B, plurality of proximal portion protruding elements 622 includes six proximal portion protruding elements, but can include one, two, three, four, five, or more than six proximal portion protruding elements in other examples. In some examples, plurality of proximal portion protruding elements 622 are distributed circumferentially around insert proximal portion 617. In some examples, plurality of proximal portion protruding elements 622 are only distributed on part of the circumference of insert proximal portion 617. In some examples, plurality of proximal portion protruding elements 622 are uniformly spaced around insert proximal portion 617. In some examples, plurality of proximal portion protruding elements 622 are not uniformly spaced around insert proximal portion 617.

Plurality of proximal cavity recesses 642 may include any suitable number of recesses. Plurality of proximal cavity recesses 642 may include a number of recesses corresponding to the plurality of proximal portion protruding elements 622 of insert proximal portion 617. As shown in the examples of FIG. 6C, plurality of proximal cavity recesses 642 includes six recesses but can include one, two, three, four, five, or more than six recesses in other examples.

In some examples, plurality of proximal portion protruding elements 622 of insert 610 and plurality of proximal cavity recesses 642 of member 630 also serve to limit proximal and distal movement of insert 610 and member 630 relative to each other. In particular, when member proximal cavity 637 receives insert proximal portion 617, plurality of proximal cavity recesses 642 receive the plurality of proximal portion protruding elements 622, which may then limit proximal and distal movement of insert 610 and member 630 relative to each other. In some examples, plurality of proximal portion protruding elements 622 are configured to deflect in order that plurality of proximal portion protruding elements 622 fit into plurality of proximal cavity recesses 642 (e.g., a snap fit).

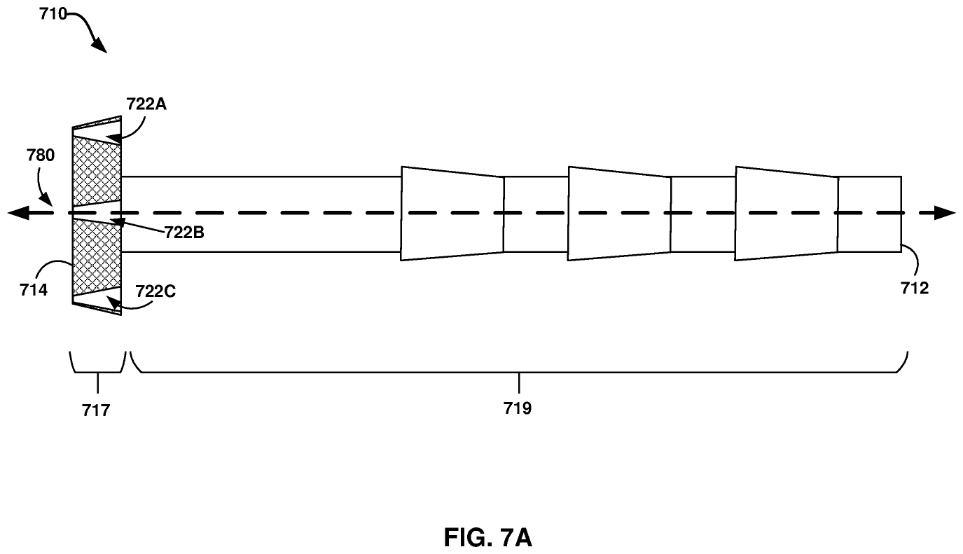

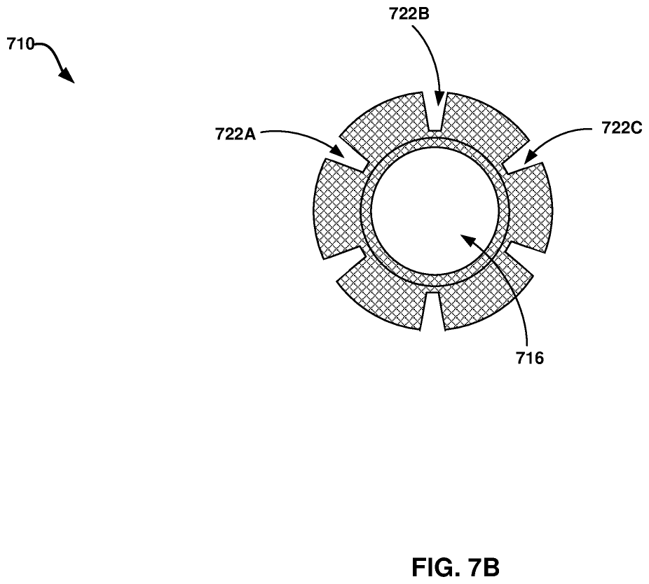

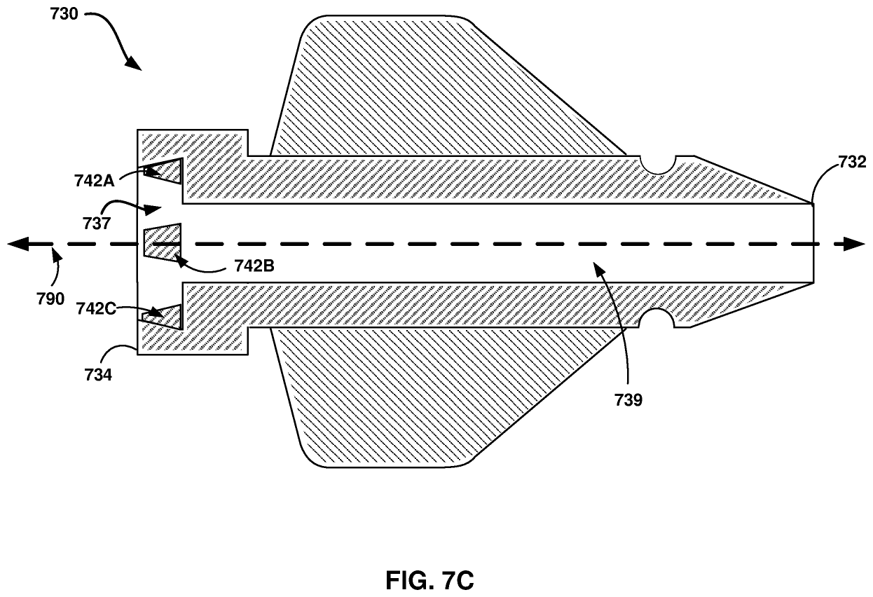

FIGS. 7A-7C illustrate various features of additional examples of the example catheter shown in FIG. 1, and may refer to the components of catheter 100 as shown in FIG. 1. Features described in relation to components shown in any of FIGS. 7A-7C may refer to features shown in other figures of FIGS. 7A-7C. FIG. 7A is a conceptual diagram illustrating a side view of an example insert 710 configured to mechanically connect a member 730 to an elongated body, such as elongated body 150. Insert 710 is another example of example insert 110 in FIG. 1, and insert 710 includes an insert proximal portion 717 and an insert distal portion 719.

FIG. 7B is a conceptual diagram illustrating a proximal end view of insert 710 and illustrates one or more features of insert 710 configured to limit movement of insert 710 relative to member 730 when insert 710 is properly positioned within in member 730 (e.g., as illustrated in FIG. 1, where insert proximal portion 717 is inserted within a member proximal cavity 737 and an insert proximal face 714 and a member proximal face 734 are aligned). Insert 710 defines an insert lumen 716.

FIG. 7C is a conceptual diagram illustrating a cross-sectional view of a member 730, the cross-section being taken along a plane parallel to a member central longitudinal axis 790 through the center of member 730. Member 730 is an example of member 130 in FIG. 1. Member 730, which may be, for example, a catheter hub, defines a member distal end 732, a member proximal face 734, a member proximal cavity 737, and a member distal lumen 739. Member distal lumen 739 is configured to receive a portion of elongated body 150. In the example of FIGS. 7A-7C, insert 710 and member 730 may be formed and function similarly to insert 210 and member 230 previously described in connection with FIG. 2, except as noted herein.

FIGS. 7A-7C illustrate another example of mating or interlocking features of insert 710 and member 730 to limit or even prevent rotation of insert 710 and member 730 relative to each other. In particular, in the example shown in FIG. 7A and FIG. 7B, insert 710 defines a plurality of proximal portion recesses 722 (shown individually in FIGS. 7A and 7B as proximal portion recesses 722A, proximal portion recesses 722B, and proximal portion recesses 722C) around the outer perimeter of insert proximal portion 717. In the example shown in FIG. 7C, member 730 includes a plurality of proximal cavity protruding elements 742 (shown individually in FIG. 7C as proximal cavity protruding element 742A, proximal cavity protruding element 742B, and proximal cavity protruding element 742C) around member proximal cavity 737. Plurality of proximal portion recesses 722 and proximal cavity protruding elements 742 are configured to mate together to limit or even prevent rotation of insert 710 relative to member 730 about an insert central longitudinal axis 780 and/or member central longitudinal axis 790 (which can be coaxial with insert central longitudinal axis 780 in some examples). That is, each proximal portion recess 722A, 722B, 722C of plurality of proximal portion recesses 722 is configured to receive a respective a respective proximal cavity protruding element 742A, 742B, 742C of proximal cavity protruding elements 742 to rotationally fix insert 710 and member 730. Similarly, plurality of proximal portion recesses 722 may be configured to receive proximal cavity protruding elements 742 to limit rotation of member 730 relative to insert 710.

In some examples, although rotation of insert 710 and member 730 relative to each other may be limited, insert 710 and member 730 may be able to rotate a small amount relative to member 730 and insert 710, respectively (e.g., less than 5 degrees, such as less than 1, 2, 3, 4, or 5 degrees) due at least in part to a small tolerance between proximal portion recesses 722 and proximal cavity protruding elements 742.

Plurality of proximal portion recesses 722 may include any suitable number of proximal portion recesses. In the example of FIGS. 7A and 7B, plurality of proximal portion recesses 722 includes six proximal portion recesses, but can include one, two, three, four, five, or more than six proximal portion recesses in other examples. In some examples, plurality of proximal portion recesses 722 are distributed circumferentially around insert proximal portion 717. In some examples, plurality of proximal portion recesses 722 are only distributed of part of the circumference of insert proximal portion 717. In some examples, plurality of proximal portion recesses 722 are uniformly spaced around insert proximal portion 717. In some examples, plurality of proximal portion recesses 722 are not uniformly spaced around insert proximal portion 717.

Plurality of proximal cavity protruding elements 742 may include any suitable number of protruding elements. Plurality of proximal cavity protruding elements 742 may include a number of protruding elements corresponding to plurality of proximal portion recesses 722 of insert proximal portion 717. As shown in the examples of FIG. 7C, plurality of proximal cavity protruding elements 742 includes six elements but can include one, two, three, four, five or more than six elements in other examples.

In some examples, plurality of proximal cavity protruding elements 742 of member 730 and plurality of proximal portion recesses 722 of insert 710 also serve to limit proximal and distal movement of insert 710 and member 730 relative to each other. In particular, when member proximal cavity 737 receives insert proximal portion 717, plurality of proximal portion recesses 722 receive plurality of proximal cavity protruding elements 742, which may then limit proximal and distal movement of insert 710 and member 730 relative to each other. In some examples, plurality of proximal cavity protruding elements 742 are configured to deflect in order that plurality of proximal cavity protruding elements 742 fit into plurality of proximal portion recesses 722 (e.g., a snap fit).

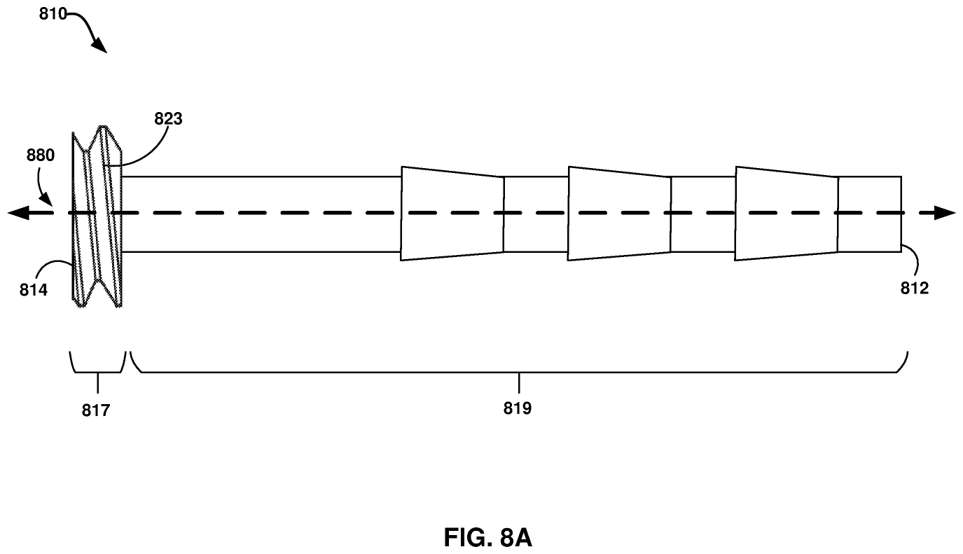

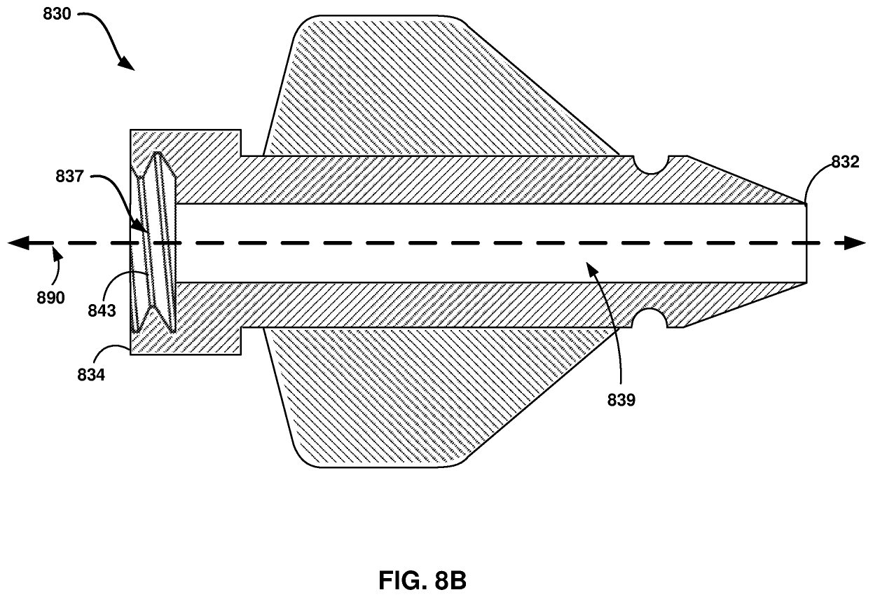

FIGS. 8A-8B illustrate various features of additional examples of the example catheter shown in FIG. 1, and may refer to the components of catheter 100 as shown in FIG. 1. Features described in relation to components shown in any of FIGS. 8A-8B may refer to features shown in other figures of FIGS. 8A-8B. FIG. 8A is a conceptual diagram illustrating a side view of an example insert 810 configured to mechanically connect a member 830 to an elongated body, such as elongated body 150. Insert 810 is another example of example insert 110 in FIG. 1, and insert 810 includes an insert proximal portion 817 and an insert distal portion 819.

FIG. 8B is a conceptual diagram illustrating a cross-sectional view of a member 830, the cross-section being taken along a plane parallel to a member central longitudinal axis 890 through the center of member 830. Member 830 is an example of member 130 in FIG. 1. Member 830, which may be, for example, a catheter hub, defines a member distal end 832, a member proximal face 834, a member proximal cavity 837, and a member distal lumen 839. Member distal lumen 839 is configured to receive a portion of elongated body 150. In the example of FIGS. 8A-8B, insert 810 and member 830 may be formed and function similarly to insert 210 and member 230 previously described in connection with FIG. 2, except as noted herein.

FIGS. 8A-8B illustrate an example of mating or interlocking features of insert 810 and member 830 to limit rotation of insert 810 and member 830 relative to each other as well as limit proximal and distal movement of insert 810 and member 830 relative to each other and elongated body 150. In particular, in the example shown in FIG. 8A, insert proximal portion 817 of insert 810 defines a first threaded portion 823. In the example shown in FIG. 8A, member proximal cavity 837 defines a second threaded portion 843. First threaded portion 823 of insert 810 and second threaded portion 843 of member 830 are configured to engage when insert 810 is inserted into member 830. In some examples, when first threaded portion 823 of insert 810 engages second threaded portion 843 of member 830, insert 810 and member 830 are limited from moving proximally and distally relative to each other. Additionally, in some examples, when first threaded portion 823 of insert 810 engages second threaded portion 843 of member 830, insert 810 and member 830 are limited from rotating relative to each other, unless a torque sufficient to overcome the interlocking threads is provided. The limited rotation of insert 810 may be rotation around an insert central longitudinal axis 880 extending between an insert distal end 812 and an insert proximal face 814. The limited rotation of member 830 may be rotation around a member central longitudinal axis 890 extending between member distal end 832 and member proximal face 834.

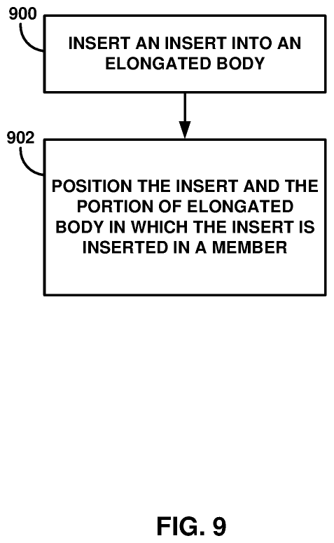

FIG. 9 is a flow diagram illustrating an example technique of assembling a catheter with a member using an insert according to one or more examples of this disclosure. The technique in FIG. 9 may be used in connection with any of the devices or systems described in connection with FIGS. 1-8, and is described with respect to catheter 100 as well as the various devices and system described in FIGS. 2A-2C. In accordance with the technique shown in FIG. 9, a user alone or with the aid of a manufacturing machine or tool, inserts insert 210 into elongated body 150 (900). As discussed above in relation to FIG. 2A, insert 210 may include insert proximal portion 217 and insert distal portion 219 and inserting insert 210 into elongated body 150 can include positioning elongated body 150 over insert distal portion 219. In some examples, insert distal portion 219 is inserted into elongated body 150 while insert proximal portion 217 is not inserted into elongated body 150 (i.e., remains outside of elongated body 150). Insert proximal portion 217 may be configured to provide a physical stop during insertion of insert distal portion 219 into elongated body 150.

Continuing with the example of FIG. 9, the technique includes positioning insert 210 and the portion of elongated body 150 in which insert 210 is inserted in member 230, such that at least insert 210 is in member proximal cavity 237 and insert 210 and the portion of elongated body 150 is in member distal lumen 239 (902). For example, a user, alone or with the aid of a manufacturing machine or tool, can retract member 230, in a proximal direction, over a portion of elongated body 150 where insert 210 has been inserted into elongated body 150. In some examples, when member proximal cavity 237 receives insert proximal portion 217, insert proximal face 214 and member proximal face 234 are aligned. Once insert 210 is in member proximal cavity and insert 210 and the portion of elongated body 150 is in member distal lumen 239, elongated body 150 is compressed between member 230 and insert 210. This may create a friction or interference fit to secure member 230 to elongated body 150.

The present disclosure includes the following non-limiting examples.
Example 1. A catheter comprising: an insert comprising an insert proximal portion and an insert distal portion, the insert proximal portion defining an insert proximal face; and a member defining a member distal lumen, a member proximal cavity, and a member proximal face, wherein the insert distal portion is configured to be inserted into an elongated body, wherein the member distal lumen is configured to receive a portion of the elongated body, wherein the member proximal cavity is configured to receive the insert proximal portion such that the insert proximal face and the member proximal face are aligned, and wherein the insert proximal portion is configured to anchor the insert within the member proximal cavity when the insert proximal portion is positioned in the member proximal cavity to limit proximal and distal movement of the insert relative to the member.
Example 2. The catheter of example 1, wherein the insert proximal portion tapers proximally to define a non-return edge, and wherein the non-return edge is configured to limit proximal movement of the member relative to the insert when the insert proximal portion is positioned in the member proximal cavity.
Example 3. The catheter of example 2, wherein the member proximal cavity tapers proximally to define a deflecting edge, and wherein when the proximal cavity receives the insert proximal portion, the deflecting edge is configured to deflect to enable insertion of the insert into the member across the non-return edge.
Example 4. The catheter of any of examples 1-3, wherein the member proximal cavity defines a first threaded portion and the insert proximal portion defines a second threaded portion, and wherein the first threaded portion and the second threaded portion are configured to engage to limit proximal and distal movement of the insert relative to the member.
Example 5. The catheter of any of examples 1-4 wherein the insert proximal portion defines a conical frustrum.
Example 6. The catheter of any of examples 1-4 wherein the insert proximal portion defines a hexagonal conical frustrum.
Example 7. The catheter of any of examples 1-6, further comprising the elongated body defining an elongated body lumen, wherein the insert defines an insert distal end and an insert lumen extending between the insert distal end and the insert proximal face, and wherein when the insert is inserted into the elongated body, the insert lumen is in fluid communication with the elongated body lumen.
Example 8. The catheter of any of examples 1-7, wherein the insert comprises a plurality of insert protrusions extending radially outward from the insert distal portion.
Example 9. The catheter of example 8, further comprising the elongated body, wherein when the insert distal portion is inserted into the elongated body and when the member distal lumen receives the portion of the elongated body, the plurality of insert protrusions is configured to compress the elongated body between the insert and the member to secure the member to the elongated body.
Example 10. The catheter of any of examples 8-9, wherein the insert and the plurality of insert protrusions are made of a single material.
Example 11. The catheter of any of examples 8-9, wherein the insert is made of a first material, wherein the plurality of insert protrusions is made of a second material, and wherein the second material is more compliant than the first material.
Example 12. The catheter of any of examples 1-7, wherein the member comprises a plurality of member protrusions extending radially inward into the member distal lumen.
Example 13. The catheter of example 12, further comprising the elongated body, wherein when the insert distal portion is inserted into the elongated body and when the member distal lumen receives the portion of the elongated body, the plurality of member protrusions is configured to compress the elongated body between the insert and the member to secure the member to the elongated body.
Example 14. The catheter of any of examples 12-13, wherein the member and the plurality of member protrusions are made of a single material.
Example 15. The catheter of any of examples 12-13, wherein the member is made of a first material, wherein the plurality of member protrusions is made of a second material, and wherein the second material is more compliant than the first material.
Example 16. The catheter of any of examples 1-15, wherein when the insert proximal portion is positioned in the member proximal cavity, the member proximal cavity and the insert proximal portion are configured to limit rotation of the insert relative to the member.
Example 17. The catheter of example 16, wherein an outer perimeter of the insert proximal portion defines a first plurality of flat sides and an inner perimeter of the member proximal cavity defines a second plurality of flat sides, the first plurality of flat sides and the second plurality of flat sides configured to engage to limit rotation of the insert relative to the member when the insert proximal portion is positioned in the member proximal cavity.
Example 18. The catheter of example 16, wherein the insert comprises a plurality of proximal portion protruding elements around the insert proximal portion and the member defines a plurality of proximal cavity recesses around the member proximal cavity, wherein the plurality of proximal cavity recesses is configured to receive the plurality of proximal portion protruding elements to limit rotation of the insert relative to the member.
Example 19. The catheter of example 16, wherein the insert defines a plurality of proximal portion recesses around the insert proximal portion and the member comprises a plurality of proximal cavity protruding elements around the member proximal cavity, and wherein the plurality of proximal portion recesses is configured to receive the plurality of proximal cavity protruding elements to limit rotation of the insert relative to the member.
Example 20. A catheter comprising: an insert comprising an insert proximal portion and an insert distal portion, the insert proximal portion defining an insert proximal face; and a member defining a member distal lumen, a member proximal cavity, and a member proximal face, wherein the insert distal portion is configured to be inserted into an elongated body, wherein the member distal lumen is configured to receive a portion of the elongated body, wherein the member proximal cavity is configured to receive the insert proximal portion such that the insert proximal face and the member proximal face are aligned, and wherein the member proximal cavity and the insert proximal portion are configured to limit rotation of the insert relative to the member when the insert proximal portion is positioned in the member proximal cavity.
Example 21. The catheter of example 20, wherein an outer perimeter of the insert proximal portion defines a first plurality of flat sides and an inner perimeter of the member proximal cavity defines a second plurality of flat sides, the first plurality of flat sides and the second plurality of flat sides configured to engage to limit rotation of the insert relative to the member when the insert proximal portion is positioned in the member proximal cavity.
Example 22. The catheter of example 20, wherein the insert comprises a plurality of proximal portion protruding elements around the insert proximal portion and the member defines a plurality of proximal cavity recesses around the member proximal cavity, wherein the plurality of proximal cavity recesses is configured to receive the plurality of proximal portion protruding elements to limit rotation of the insert relative to the member.
Example 23. The catheter of example 20, wherein the insert defines a plurality of proximal portion recesses around the insert proximal portion and the member comprises a plurality of proximal cavity protruding elements around the member proximal cavity, and wherein the plurality of proximal portion recesses is configured to receive the plurality of proximal cavity protruding elements to limit rotation of the insert relative to the member.
Example 24. The catheter of any of examples 20-23, wherein when the insert proximal portion is positioned in the member proximal cavity, the insert proximal portion is configured to anchor the insert within the member proximal cavity to limit proximal and distal movement of the insert relative to the member.
Example 25. The catheter of any of examples 20-24, wherein the insert proximal portion tapers proximally to define a non-return edge, and wherein the non-return edge is configured to limit proximal movement of the member relative to the insert when the insert proximal portion is positioned in the member proximal cavity.
Example 26. The catheter of example 25, wherein the member proximal cavity tapers proximally to define a deflecting edge, and wherein when the proximal cavity receives the insert proximal portion, the deflecting edge is configured to deflect to enable insertion of the insert into the member across the non-return edge.
Example 27. The catheter of any of examples 20-26, wherein the member proximal cavity defines a first threaded portion, and the insert proximal portion defines a second threaded portion.
Example 28. The catheter of any of examples 20-27 wherein the insert proximal portion comprises a conical frustrum.
Example 29. The catheter of any of examples 20-28 wherein the insert proximal portion comprises a hexagonal conical frustrum.
Example 30. The catheter of any of examples 20-29, further comprising the elongated body defining an elongated body lumen, wherein the insert defines an insert distal end, and an insert lumen extending between the insert distal end and the insert proximal face, wherein when the insert is inserted into the elongated body, the insert lumen is in fluid communication with the elongated body lumen.
Example 31. The catheter of any of examples 20-30, wherein the insert distal portion comprises a plurality of insert protrusions extending radially outward from the insert distal portion.
Example 32. The catheter of example 31, further comprising the elongated body, wherein when the insert distal portion is inserted into the elongated body and when the member distal lumen receives the portion of the elongated body, the plurality of insert protrusions is configured to compress the elongated body between the insert and the member to secure the member to the elongated body.
Example 33. The catheter of any of examples 31-32, wherein the insert and the plurality of insert protrusions are made of a single material.
Example 34. The catheter of any of examples 31-32, wherein the insert is made of a first material, wherein the plurality of insert protrusions is made of a second material, and wherein the second material is more compliant than the first material.
Example 35. The catheter of any of examples 20-30, wherein the member comprises a plurality of member protrusions extending radially inward into the member distal lumen.
Example 36. The catheter of example 35, further comprising the elongated body, wherein when the insert distal portion is inserted into the elongated body and when the member distal lumen receives the portion of the elongated body, the plurality of member protrusions is configured to compress the elongated body between the insert and the member to secure the member to the elongated body.
Example 37. The catheter of any of examples 35-36, wherein the member and the plurality of member protrusions are made of a single material.
Example 38. The catheter of any of examples 35-36, wherein the member is made of a first material, wherein the plurality of member protrusions is made of a second material, and wherein the second material is more compliant than the first material.
Example 39. The catheter of any of examples 1-38, wherein the member comprises a first member piece and a second member piece, the first member piece extending between the member proximal face and a member distal end and the second member piece extending between the member proximal face and a member distal end, wherein the first member piece and the second member piece are configured to be coupled together.
Example 40. The catheter of any of examples 1-39, wherein the member is a hub and the elongated body is a catheter shaft.
Example 41. The catheter of any of examples 1-40, wherein no adhesive is used to connect the insert and the member.
Example 42. A method comprising: inserting an insert into an elongated body, the insert comprising an insert proximal portion and an insert distal portion, the insert proximal portion defining an insert proximal face; and positioning the insert, and a portion of the elongated body where the insert is inserted, into a member, wherein the member defines a member distal lumen, a member proximal cavity, and a member proximal face, wherein the insert distal portion is configured to be inserted into the elongated body, wherein the member distal lumen is configured to receive a portion of the elongated body, wherein the member proximal cavity is configured to receive the insert proximal portion, and wherein when the member proximal cavity receives the insert proximal portion, the insert proximal face and the member proximal face are aligned.
Example 43. The method of example 42, wherein positioning the insert and the portion of the elongated body where the insert is inserted into a member comprises retracting the member, in a proximal direction, over the portion of the elongated body where the insert has been inserted into the elongated body.

Various examples of the disclosure have been described. Any combination of the described systems, operations, or functions is contemplated. These and other examples are within the scope of the claims.

1. A catheter comprising:
an insert comprising an insert proximal portion and an insert distal portion, the insert proximal portion defining an insert proximal face; and
a member defining a member distal lumen, a member proximal cavity, and a member proximal face,
wherein the insert distal portion is configured to be inserted into an elongated body,
wherein the member distal lumen is configured to receive a portion of the elongated body,
wherein the member proximal cavity is configured to receive the insert proximal portion such that the insert proximal face and the member proximal face are aligned, and
wherein the insert proximal portion is configured to anchor the insert within the member proximal cavity when the insert proximal portion is positioned in the member proximal cavity to limit proximal and distal movement of the insert relative to the member.

2. The catheter of claim 1, wherein the insert proximal portion tapers proximally to define a non-return edge, and wherein the non-return edge is configured to limit proximal movement of the member relative to the insert when the insert proximal portion is positioned in the member proximal cavity.

3. The catheter of claim 2, wherein the member proximal cavity tapers proximally to define a deflecting edge, and wherein when the member proximal cavity receives the insert proximal portion, the deflecting edge is configured to deflect to enable insertion of the insert into the member across the non-return edge.

4. The catheter of claim 1, wherein the member proximal cavity defines a first threaded portion and the insert proximal portion defines a second threaded portion, and wherein the first threaded portion and the second threaded portion are configured to engage to limit proximal and distal movement of the insert relative to the member.

5. The catheter of claim 1, wherein the insert proximal portion defines a conical frustrum.

6. The catheter of claim 1, further comprising the elongated body defining an elongated body lumen,
wherein the insert defines an insert distal end and an insert lumen extending between the insert distal end and the insert proximal face, and
wherein when the insert is inserted into the elongated body, the insert lumen is in fluid communication with the elongated body lumen.

7. The catheter of claim 1, wherein the insert comprises a plurality of insert protrusions extending radially outward from the insert distal portion.

8. The catheter of claim 7, further comprising the elongated body, wherein when the insert distal portion is inserted into the elongated body and when the member distal lumen receives the portion of the elongated body, the plurality of insert protrusions is configured to compress the elongated body between the insert and the member to secure the member to the elongated body.

9. The catheter of claim 7, wherein the insert and the plurality of insert protrusions are made of a single material.

10. The catheter of claim 7, wherein the insert is made of a first material, wherein the plurality of insert protrusions is made of a second material, and wherein the second material is more compliant than the first material.

11. The catheter of claim 1, wherein the member comprises a plurality of member protrusions extending radially inward into the member distal lumen.

12. The catheter of claim 11, further comprising the elongated body, wherein when the insert distal portion is inserted into the elongated body and when the member distal lumen receives the portion of the elongated body, the plurality of member protrusions is configured to compress the elongated body between the insert and the member to secure the member to the elongated body.

13. The catheter of claim 11, wherein the member is made of a first material, wherein the plurality of member protrusions is made of a second material, and wherein the second material is more compliant than the first material.

14. The catheter of claim 1, wherein when the insert proximal portion is positioned in the member proximal cavity, the member proximal cavity and the insert proximal portion are configured to limit rotation of the insert relative to the member.

15. The catheter of claim 14, wherein an outer perimeter of the insert proximal portion defines a first plurality of flat sides and an inner perimeter of the member proximal cavity defines a second plurality of flat sides, the first plurality of flat sides and the second plurality of flat sides configured to engage to limit rotation of the insert relative to the member when the insert proximal portion is positioned in the member proximal cavity.

16. A catheter comprising:
an insert comprising an insert proximal portion and an insert distal portion, the insert proximal portion defining an insert proximal face; and
a member defining a member distal lumen, a member proximal cavity, and a member proximal face,
wherein the insert distal portion is configured to be inserted into an elongated body,
wherein the member distal lumen is configured to receive a portion of the elongated body,
wherein the member proximal cavity is configured to receive the insert proximal portion such that the insert proximal face and the member proximal face are aligned, and
wherein the member proximal cavity and the insert proximal portion are configured to limit rotation of the insert relative to the member when the insert proximal portion is positioned in the member proximal cavity.

17. The catheter of claim 16, further comprising the elongated body defining an elongated body lumen,
wherein the insert defines an insert distal end, and an insert lumen extending between the insert distal end and the insert proximal face, and
wherein when the insert is inserted into the elongated body, the insert lumen is in fluid communication with the elongated body lumen.

18. The catheter of claim 16, wherein no adhesive is used to connect the insert and the member.

19. A method comprising:
inserting an insert into an elongated body, the insert comprising an insert proximal portion and an insert distal portion, the insert proximal portion defining an insert proximal face; and
positioning the insert, and a portion of the elongated body where the insert is inserted, into a member, wherein the member defines a member distal lumen, a member proximal cavity, and a member proximal face,
wherein the insert distal portion is configured to be inserted into the elongated body,
wherein the member distal lumen is configured to receive the portion of the elongated body,
wherein the member proximal cavity is configured to receive the insert proximal portion, and
wherein when the member proximal cavity receives the insert proximal portion, the insert proximal face and the member proximal face are aligned.

20. The method of claim 19, wherein positioning the insert and the portion of the elongated body where the insert is inserted into the member comprises retracting the member, in a proximal direction, over the portion of the elongated body where the insert has been inserted into the elongated body.
